(19) United States
(12) Patent Application Publication
(10) Pub. No.: US 2016/0280731 A1
(43) Pub. Date: Sep. 29, 2016

(54) CRYSTALLINE FORMS B, C, and D OF CANAGLIFLOZIN

(71) Applicant: CRYSTAL PHARMATECH CO., LTD, Suzhou (CN)

(72) Inventors: Minhua CHEN, Suzhou (CN); Yanfeng ZHANG, Suzhou (CN); Yi ZHAO, Suzhou (CN); Ye ZHAO, Suzhou (CN); Xiaoyu ZHANG, Suzhou (CN); Chaohui YANG, Suzhou (CN); Fei LU, Suzhou (CN)

(73) Assignee: CRYSTAL PHARMATECH CO., LTD, Suzhou (CN)

(21) Appl. No.: 15/035,751

(22) PCT Filed: Nov. 11, 2014
(86) PCT No.: PCT/IB2014/003013

§ 371 Date: May 10, 2016

(30) Foreign Application Priority Data
Nov. 11, 2013 (CN) 201310556655.2
Nov. 27, 2013 (CN) 201310617597.X
Oct. 14, 2014 (CN) 201410542984.6
Oct. 29, 2014 (CN) 201410593413.5

(51) Int. Cl. C07H 7/06

(52) U.S. Cl. CPC C07H 7/06; C07B 2200/13

(57) ABSTRACT

Novel crystalline Forms B, C, and D of canagliflozin, processes for their preparation, pharmaceutical compositions comprising these new Forms, and use of them for treating or delaying progression or onset of diseases or disorders related to activity of sodium-glucose transport proteins are disclosed. These novel Forms were characterized by X-ray powder diffraction, differential scanning calorimetry, and other techniques. They can be readily prepared and are suitable for preparation of solid dosage forms owing to their ease of handling and superior pharmacological properties.

### CROSS REFERENCE TO RELATED APPLICATIONS

This application is a U.S. national phase application of PCT/M2014/003013 filed on Nov., 11, 2014, and claims priority under 35 U.S.C. §119(a) to Chinese Patent Application No. 201310556655.2, filed on Nov. 11, 2013, and Chinese Patent Application No. 201310617597.X, filed on Nov. 27, 2013, both of which are herein incorporated by reference in their entireties.

### FIELD OF THE INVENTION

This invention relates to novel crystalline forms of canagliflozin, or hydrates thereof, and their pharmaceutical compositions, methods of preparation, and methods of uses.

### BACKGROUND OF THE INVENTION

Canagliflozin is an inhibitor of the sodium-glucose transport proteins (SGLT2), the transporter responsible for reabsorbing the majority of glucose filtered by the kidney, approved by the United States Food and Drug Administration (FDA) for the treatment of type II diabetes. Canagliflozin lowers blood sugar by causing the kidneys to remove more glucose from the urine. Canagliflozin is a glucopyranoside derivative, namely (2S,3R,4R,5S,6R)-2-{3-[5-[4-fluoro-phenyl)-thiophen-2-ylmethyl]-4-methyl-phenyl -}-6-hydroxymethyl-tetrahydro-pyran-3,4,5-triol, having the structure of Formula I:

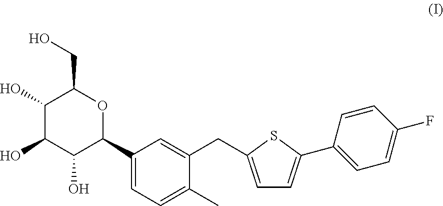 [SMILES: Cc1ccc([C@@H]2O[C@H](CO)[C@@H](O)[C@H](O)[C@H]2O)cc1Cc1ccc(-c2ccc(F)cc2)s1]

Polymorphism is the ability of a substance to exist as two or more crystalline phases that have different arrangement and/or conformations of the molecule in the crystal lattice. Different crystalline forms of a compound may exhibit different solubility and dissolution rate, thus affecting properties of an active pharmaceutical ingredient, such as bioavailability and/or even efficacy, but in general it is not possible to predict how many crystalline forms or polymorphs may exist for any specific compounds or which crystalline form would possess superior properties; nor would it be possible to predict conditions by which any specific crystalline form of a compound could be prepared. Hence, the discovery of a new crystalline form of a pharmaceutically useful compound plays a crucial role during the pharmaceutical development. For example, for a poorly soluble compound, discovery of a new crystalline form with desired solubility may provide an opportunity to improve the performance of the active pharmaceutical ingredient.

Canagliflozin has been reported to exist as a crystalline hemihydrate form (WO2008069327A1), and has been prepared in a crystalline form using a complex crystallization process under protection of argon (WO2009035969A1).

In addition, amorphous canagliflozin and its cocrystals with amino acids, such as L-proline, D-proline and L-phenylalanine, have also been reported (WO2013064909A2).

New crystalline forms of canagliflozin, in particular stable polymorphs with superior pharmacological activities suitable for formulation, and convenient methods to prepare them remain a great need.

### SUMMARY OF THE INVENTION

The present inventors surprisingly discovered novel crystalline forms of canagliflozin, which have improved solubility, among others, over the known crystalline hemihydrate form reported in WO2008069327A1 and desired pharmacological properties useful for pharmaceutical development. These crystalline forms of canagliflozin can be prepared in environmentally friendly solvent systems.

In one aspect, the present invention provides crystalline forms of canagliflozin or hydrate thereof, designated as Forms B, C, and D, respectively.

In another aspect, the present invention provides processes for preparation of canagliflozin Forms B, C, and D, respectively.

In another aspect, the present invention provides pharmaceutical compositions comprising any of the crystalline Forms B, C, and D of canagliflozin, or a hydrate thereof, and a pharmaceutically acceptable carrier.

In another aspect, the present invention provides methods of using crystalline Form B, C, or D of canagliflozin or hydrate, or a combination thereof, in the manufacture of a medicament for treating or delaying the progression or onset of a disease or disorder in connection with activity of a sodium-glucose transport (SGLT) protein.

In another aspect, the present invention provides methods of treating or delaying the progression or onset of a disease or disorder in connection with activity of a sodium-glucose transport (SGLT) protein, comprising administering to a subject in need thereof a pharmaceutical composition comprising any one of crystalline Forms B, C, and D of canagliflozin or hydrate, or a combination thereof.

The diseases and disorders in connection with the activity of a sodium-glucose transport (SGLT) protein include, but are not limited to, diabetes mellitus, diabetic retinopathy, diabetic neuropathy, diabetic nephropathy, delayed wound healing, insulin resistance, hyperglycemia, hyperinsulinemia, elevated blood levels of fatty acids, elevated blood levels of glycerol, hyperlipidemia, obesity, hypertriglyceridemia, Syndrome X, diabetic complications, atherosclerosis, and hypertension.

Other aspects and embodiments of the present invention will be further illustrated in the following description and examples.

### BRIEF DESCRIPTION OF THE DRAWINGS

FIG. 1 shows a powder X-ray diffraction (MUD) pattern of crystalline Form B.

FIG. 2 shows a differential scanning calorimetry (DSC) thermogram of crystalline Form B.

FIG. 3 shows a thermal gravimetric analysis (TGA) thermogram of crystalline Form B.

FIG. 4 shows comparison of the XRPD pattern of Form B before storage (top pattern) and the XRPD pattern of Form B after being stored under 25° C./60% RH for 30 days (bottom pattern).

FIG. 5 shows comparison of the XRPD pattern of Form B before storage (top pattern) and the XRPD pattern of Form B after being stored under 40° C./75% RH for 30 days (bottom pattern).

FIG. 6 shows a powder X-ray diffraction (XRPD) pattern of crystalline Form C.

FIG. 7 shows a differential scanning calorimetric (DSC) thermogram of crystalline Form C.

FIG. 8 shows a thermal gravimetric analysis (TGA) thermogram of crystalline Form C.

FIG. 9 shows a powder X-ray diffraction (XRPD) pattern of crystalline Form D.

### DETAILED DESCRIPTION OF THE INVENTION

The present invention is based on a surprising discovery that canagliflozin can exist in different crystalline Forms, and these Forms can be prepared readily from environmentally friendly solvent systems using relatively simple processes. These new Forms were discovered, surprisingly, to have better solubility than the known crystalline hemihydrate form, and thus may make them better suited for preparation of pharmaceutical compositions, especially solid formulations and dosage forms.

In an aspect, the present invention provides a crystalline form of canagliflozin, designated as Form B.

In one embodiment, the crystalline Form B is characterized by a powder X-ray diffraction pattern comprising the following 2θ values measured using CuKα radiation: 6.3°±0.2°, 9.4°±0.2° and 12.6°±0.2°.

In another embodiment, the crystalline Form B is characterized by a powder X-ray diffraction pattern further comprising the following 2θ values measured using CuKα radiation: 11.7°±0.2°, 16.9°±0.2°, and 19.9°±0.2°.

In another embodiment, the crystalline Form B is characterized by a powder X-ray diffraction pattern further comprising the following 2θ values measured using CuKα radiation: 18.2°±0.2°, 22.3°±0.2°, 24.4°±0.2°, and 28.9°±0.2°.

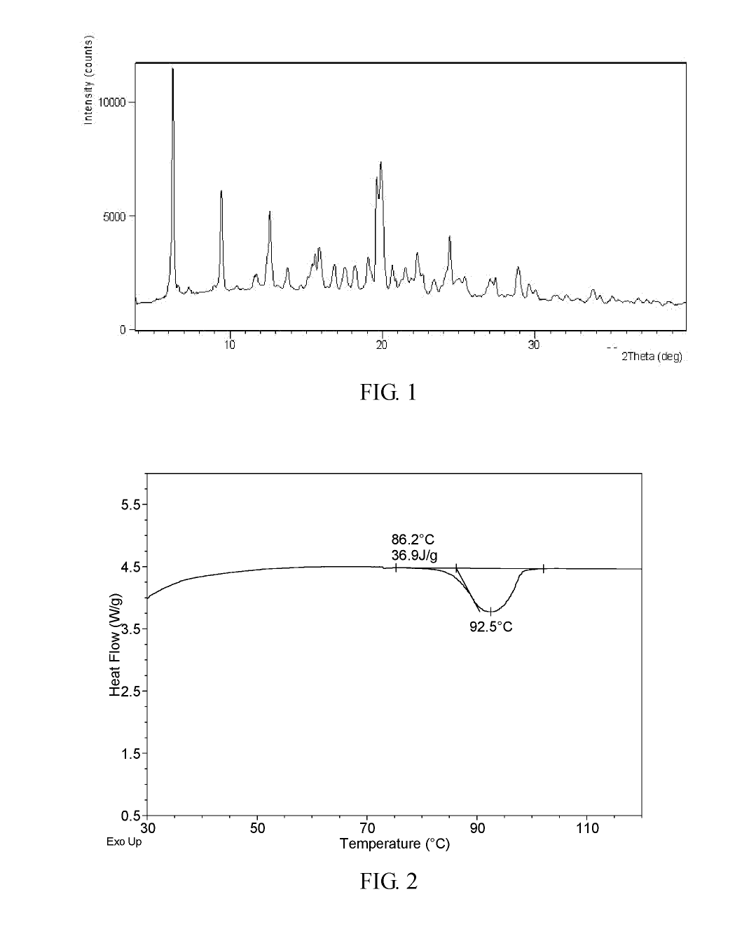

In another embodiment, the crystalline Form B has an X-ray diffraction pattern substantially as shown in FIG. 1.

In another embodiment, the crystalline Form B has a differential scanning calorimetry thermogram substantially as shown in FIG. 2, which exhibits an endothermic peak at about 86.2° C.

In another embodiment, the crystalline Form B has a thermal gravimetric analysis thermogram substantially as shown in FIG. 3, which exhibits about 6.9% weight loss when heated up to 113° C. While not intended to be limiting, based on the TGA data, the Form B appears to be, or is close to be, a dihydrate form (1.83 molecules H₂O per molecule) of canagliflozin, assuming all the weight loss is owing to the loss of water molecules co-crystallized with the compound.

In another aspect, the present invention provides a process for preparation of canagliflozin Form B, which comprises dissolving canagliflozin in a mixed solvent system comprising water and an organic solvent, filtering the solution and crystallizing the compound from the solution by slow evaporation or cooling.

In some embodiments, said mixed solvent system has a volume ratio of water to organic solvent in the range from about 1:10 to about 10:1. In a preferred embodiment, the volume ratio is about 1:1.

In some embodiments, said organic solvents include, but are not limited to, methanol, ethanol, 2-propanol, acetonitrile, acetone, and tetrahydrofuran. In a preferred embodiment, the organic solvent is ethanol or tetrahydrofuran.

In another aspect, the present invention provides a crystalline form of canagliflozin or hydrate thereof, designated as Form C.

In one embodiment, the crystalline Form C is characterized by a powder X-ray diffraction pattern comprising the following 2θ values measured using CuKα radiation: 6.5°±0.2°, 9.8°±0.2°, and 16.4°±0.2°.

In another embodiment, the powder X-ray diffraction pattern of Crystalline Form C further comprises the following 2θ values measured using CuKα radiation: 13.1°±0.2°, 19.8°±0.2°, and 23.7°±0.2°.

In another embodiment, the powder X-ray diffraction pattern of Crystalline Form C further comprises the following 2θ values measured using CuKα radiation: 17.1°±0.2°, 19.5°±0.2°, 25.2°±0.2°, and 26.5°±0.2°.

In another embodiment, the crystalline Form C has an X-ray diffraction pattern substantially as depicted in FIG. 6.

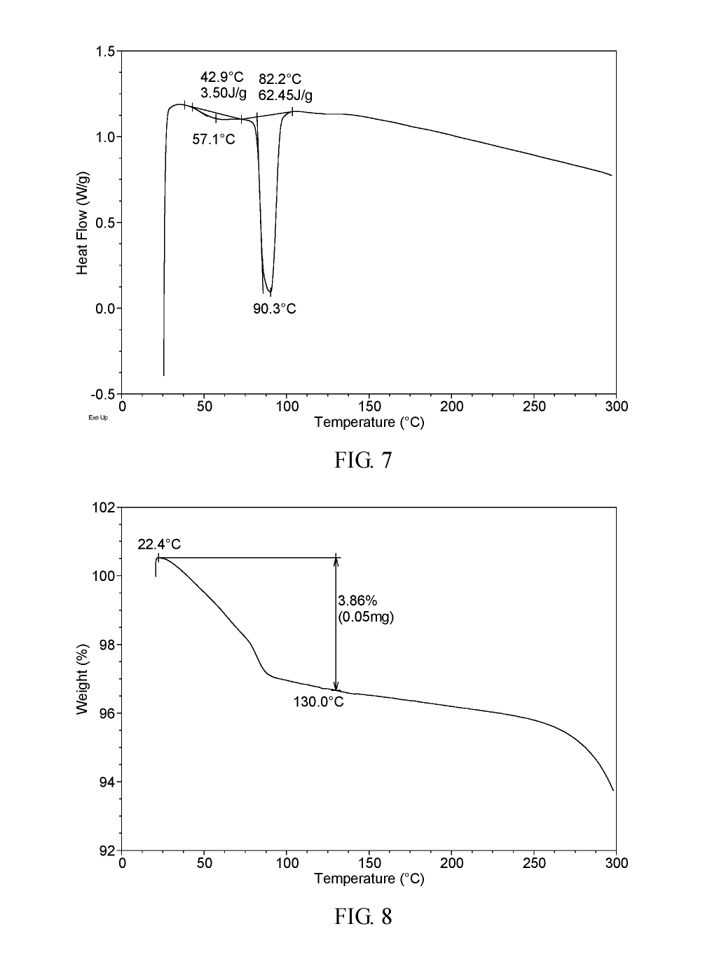

In another embodiment, the crystalline Form C has a differential scanning calorimetry thermogram comprising two endothermic peaks at about 42.9° C. and about 82.2° C., respectively, as shown in FIG. 7.

In another embodiment, the crystalline Form C of canagliflozin has a thermal gravimetric analysis (TGA) thermogram comprising about 3.86% weight loss up to 130° C., as shown in FIG. 8. While not intended to be limiting, based on the TGA data, the Form C appears to be, or is close to be, a monohydrate form (1.0 molecule H₂O per molecule) of canagliflozin, assuming all the weight loss is owing to the loss of water molecules co-crystallized with the compound.

In another aspect, the present invention provides processes for preparation of canagliflozin Form C, which comprises forming a solution of canagliflozin in a mixed solvent system of water and tetrahydrofuran, evaporating the solution to dryness, and crystallizing said Form C by sweeping the solids using air or N₂.

In another aspect, the present invention provides a crystalline form of canagliflozin, designated as Form D.

In one embodiment, the crystalline Form D is characterized by a powder X-ray diffraction pattern comprising the following 2θ values measured using CuKα radiation: 6.8°±0.2°, 13.6°±0.2°, and 20.5°±0.2°.

In another embodiment, the powder X-ray diffraction pattern of crystalline Form D further comprises the following 2θ values measured using CuKα radiation: 17.1°±0.2°, 19.2°±0.2°, and 22.9°±0.2°.

In another embodiment, the powder X-ray diffraction pattern of crystalline Form D further comprises the following 2θ values measured using CuKα radiation: 10.2°±0.2°, 16.5°±0.2°, 18.5°±0.2°, and 24.4°±0.2°.

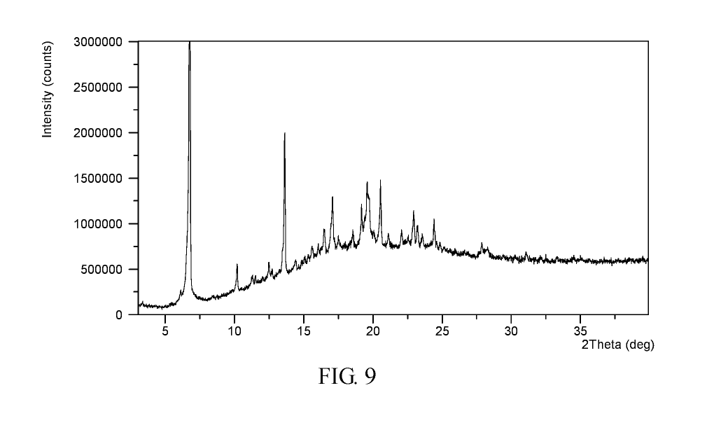

In another embodiment, the crystalline Form D has an X-ray diffraction pattern substantially as shown in FIG. 9.

In another aspect, the present invention provides a process for preparation of canagliflozin Form D, which comprises crystallizing said Form D by heating Form C to a temperature in the range of about 50° C-90° C.

In another aspect, the present invention comprises a pharmaceutical composition comprising any of crystalline Forms B, C, or D, or a combination thereof, and a pharmaceutically acceptable carrier. Compositions of the present invention may further comprise one or more other pharmaceutically acceptable excipients.

Form B, C or D of canagliflozin or hydrates thereof, together with one or more pharmaceutically acceptable excipients, of the present invention may be further formulated as: solid oral dosage forms such as, but not limited to, powders, granules, pellets, tablets, and capsules; liquid oral dosage forms such as, but not limited to, syrups, suspensions, dispersions, and emulsions; and injectable preparations such as, but not limited to, solutions, dispersions, and freeze dried compositions. Formulations may be in the forms of immediate release, delayed release or modified release. Further, immediate release compositions may be conventional, dispersible, chewable, mouth dissolving, or flash melt preparations; and modified release compositions may comprise hydrophilic or hydrophobic, or combinations of hydrophilic and hydrophobic, release rate controlling substances to form matrix or reservoir, or combination of matrix and reservoir systems.

The compositions may be prepared using techniques such as direct blending, dry granulation, wet granulation, and extrusion and spheronization. Compositions may be presented as uncoated, film coated, sugarcoated, powder coated, enteric coated, or modified release coated.

In another aspect, the present invention provides a method for treating or delaying the progression or onset of diabetes mellitus, diabetic retinopathy, diabetic neuropathy, diabetic nephropathy, delayed wound healing, insulin resistance, hyperglycemia, hyperinsulinemia, elevated blood levels of fatty acids, elevated blood levels of glycerol, hyperlipidemia, obesity, hypertriglyceridemia, Syndrome X, diabetic complications, atherosclerosis, or hypertension, which comprises administering a subject in need of treatment a therapeutically effective amount of canagliflozin comprising crystalline Form B, Form C, or Form D, or a combination thereof.

### EXAMPLES

### X-Ray Powder Diffraction (XRPD)

Analytical Instrument: Panalytical Empyrean. The X-ray powder diffractogram was determined by mounting a sample of the crystalline material on a Si single crystal low-background holder and spreading out the sample into a thin layer with the aid of a microscope slide. The 2θ position was calibrated against Panalytical 640 Si powder standard. The sample irradiated with X-rays generated by a copper long-fine focus tube operated at 45 kV and 40 mA with a wavelength of Kα1=1.540589 angstroms and Kα2=1.544426 angstroms (Kα1/Kα2 intensity ratio is 0.50). The collimated X-ray source was passed through a programmed divergence slit set at 10 mm and the reflected radiation directed through a 5.5 mm anti-scattering slit. The sample was exposed for 16.3 seconds per 0.013°2-theta increment (continuous scan mode) over the range 3 degrees to 40 degrees 2-theta in theta-theta mode. The running time was 3 minutes and 57 seconds. The instrument was equipped with an RTMS detector (X′Celerator). Control and data capture was accomplished by means of a Dell Optiplex 780 XP operating with data collector software.

Persons skilled in the art of X-ray powder diffraction will realize that the relative intensity of peaks can be affected by, for example, grains above 30 microns in size and non-unitary aspect ratios that may affect analysis of samples. The skilled person will also realize that the position of reflections can be affected by the precise height at which the sample sits in the diffractometer and the zero calibration of the diffractometer. The surface planarity of the sample may also have a small effect. Hence, the diffraction pattern data presented are not to be taken as absolute values.

### Differential Scanning Calorimetry (DSC)

Analytical Instrument: TA Instruments Q2000 DSC.

Heating rate: 10° C. per minute.

Purge gas: nitrogen

### Thermal Gravimetric Analysis(TGA)

Analytical Instrument: TA Instruments Q5000 TGA.

Heating rate: 10° C. per minute.

Purge gas: nitrogen.

### Example 1

### Preparation of Canagliflozin Crystalline Form B

To a solvent system of ethanol/water (1:1, v/v) was added 10.6 mg of canagliflozin, and then the suspension was filtered using a 0.45 μm Nylon filter. The solution was evaporated slowly under ambient conditions until solids precipitated from the solution. The solids were isolated to obtain crystalline Form B of canagliflozin, which was analyzed by XRPD, DSC, and TGA. The XRPD data of the Form B obtained in this Example are listed in Table 1.

The XRPD diagram, DSC thermogram, and TGA thermogram of Form B obtained from this Example are displayed in FIGS. 1-3, respectively.

TABLE 1

| 2 theta | d spacing | intensity |
|---|---|---|
| 3.79 | 23.34 | 4.05 |
| 6.26 | 14.11 | 100.00 |
| 6.66 | 13.26 | 4.82 |
| 7.33 | 12.06 | 4.32 |
| 9.43 | 9.38 | 47.40 |
| 10.55 | 8.39 | 4.36 |
| 11.80 | 7.50 | 12.13 |
| 12.60 | 7.02 | 34.31 |
| 13.78 | 6.42 | 8.24 |
| 14.68 | 6.04 | 6.80 |
| 15.05 | 5.89 | 6.09 |
| 15.33 | 5.78 | 6.80 |
| 15.79 | 5.61 | 17.32 |
| 16.88 | 5.25 | 8.28 |
| 17.49 | 5.07 | 10.62 |
| 17.74 | 5.00 | 11.46 |
| 18.20 | 4.88 | 9.95 |
| 18.36 | 4.83 | 7.09 |
| 18.85 | 4.71 | 14.89 |
| 19.01 | 4.67 | 14.09 |
| 19.66 | 4.52 | 33.63 |
| 19.90 | 4.46 | 53.58 |
| 20.69 | 4.29 | 6.49 |
| 21.19 | 4.19 | 6.82 |
| 21.43 | 4.15 | 10.20 |
| 21.91 | 4.06 | 6.09 |
| 22.24 | 3.99 | 12.76 |
| 22.33 | 3.99 | 13.13 |
| 22.60 | 3.93 | 6.63 |
| 23.39 | 3.80 | 6.03 |
| 23.90 | 3.72 | 6.23 |
| 24.17 | 3.68 | 15.01 |
| 24.43 | 3.64 | 11.25 |
| 24.88 | 3.58 | 6.69 |
| 25.40 | 3.50 | 8.39 |
| 26.58 | 3.35 | 4.50 |
| 26.81 | 3.32 | 7.40 |
| 27.07 | 3.29 | 10.62 |
| 27.44 | 3.25 | 5.49 |
| 28.24 | 3.16 | 2.92 |
| 28.82 | 3.10 | 5.74 |
| 29.11 | 3.07 | 4.51 |
| 29.57 | 3.02 | 3.68 |
| 30.03 | 2.97 | 4.73 |
| 31.27 | 2.86 | 2.93 |
| 31.99 | 2.80 | 3.83 |
| 32.71 | 2.74 | 2.86 |
| 33.62 | 2.66 | 3.06 |
| 34.34 | 2.61 | 1.53 |
| 34.97 | 2.56 | 1.92 |

### Example 2

### Preparation of Canagliflozin Form B

To a mixture of water/tetrahydrofuran (1:1, v/v) was added 52.3 mg of canagliflozin, and then the solution was filtered using a 0.45 μm Nylon filter. The solvent was slowly evaporated under ambient conditions until solids precipitated from the solution. The solids were isolated to obtain crystalline Form B, which was analyzed by XRPD. The XRPD data of the Form B prepared in this Example are listed in Table 2.

TABLE 2

| 2 theta | d spacing | intensity |
|---|---|---|
| 3.15 | 28.03 | 0.87 |
| 6.28 | 14.07 | 100.00 |
| 6.67 | 13.25 | 2.06 |
| 7.35 | 12.03 | 1.26 |
| 8.98 | 9.85 | 2.45 |
| 9.28 | 9.53 | 9.78 |
| 9.41 | 9.39 | 98.37 |
| 11.51 | 7.69 | 3.75 |
| 11.70 | 7.57 | 7.40 |
| 12.35 | 7.17 | 4.89 |
| 12.55 | 7.05 | 58.47 |
| 13.66 | 6.48 | 2.90 |
| 14.57 | 6.08 | 1.78 |
| 14.92 | 5.94 | 2.24 |
| 15.20 | 5.83 | 2.99 |
| 15.52 | 5.71 | 3.08 |
| 15.70 | 5.64 | 17.35 |
| 16.71 | 5.31 | 2.87 |
| 17.54 | 5.06 | 6.12 |
| 17.97 | 4.94 | 3.81 |
| 18.23 | 4.87 | 6.04 |
| 18.86 | 4.70 | 26.22 |
| 18.91 | 4.70 | 19.63 |
| 19.63 | 4.52 | 7.34 |
| 19.74 | 4.49 | 7.94 |
| 19.88 | 4.46 | 6.97 |
| 20.03 | 4.43 | 5.43 |
| 20.63 | 4.30 | 2.44 |
| 21.26 | 4.18 | 5.20 |
| 21.81 | 4.07 | 2.18 |
| 22.02 | 4.03 | 6.43 |
| 22.36 | 3.97 | 3.47 |
| 23.36 | 3.81 | 2.21 |
| 23.71 | 3.75 | 3.25 |
| 23.93 | 3.72 | 3.48 |
| 24.14 | 3.68 | 4.90 |
| 24.54 | 3.62 | 2.35 |
| 24.76 | 3.59 | 5.06 |
| 25.12 | 3.54 | 3.14 |
| 25.24 | 3.53 | 3.41 |
| 26.38 | 3.38 | 1.51 |
| 26.81 | 3.32 | 11.73 |
| 27.08 | 3.29 | 3.34 |
| 28.12 | 3.17 | 1.06 |
| 28.36 | 3.14 | 1.22 |
| 28.70 | 3.11 | 1.93 |
| 29.05 | 3.07 | 0.80 |
| 29.85 | 2.99 | 1.49 |
| 31.18 | 2.87 | 1.59 |
| 31.83 | 2.81 | 6.71 |

### Example 3

### Preparation of Canagliflozin Form B

In 10 mL of tetrahydrofuran was dissolved 508.1 mg of canagliflozin, followed by addition of 30 mL of water. The solution was heated to and equilibrated at 60° C., then slowly cooled to 20° C. The solution was stirred overnight with cap off to obtain crystalline Form B. The XRPD data of the Form B obtained from this Example are listed in Table 3.

TABLE 3

| 2 theta | d spacing | intensity |
|---|---|---|
| 6.21 | 14.23 | 35.70 |
| 6.75 | 13.10 | 9.67 |
| 7.37 | 12.00 | 6.84 |
| 9.00 | 9.82 | 11.90 |
| 9.55 | 9.26 | 22.48 |
| 10.60 | 8.34 | 7.40 |
| 11.71 | 7.55 | 21.45 |
| 12.42 | 7.13 | 26.51 |
| 12.58 | 7.04 | 33.61 |
| 13.75 | 6.44 | 19.38 |
| 15.29 | 5.80 | 24.45 |
| 15.58 | 5.69 | 24.53 |
| 15.88 | 5.58 | 27.97 |
| 16.81 | 5.28 | 29.57 |
| 17.55 | 5.05 | 32.37 |
| 18.05 | 4.92 | 23.60 |
| 18.29 | 4.85 | 20.60 |
| 18.95 | 4.68 | 30.34 |
| 19.68 | 4.51 | 76.97 |
| 19.82 | 4.48 | 100.00 |
| 20.03 | 4.43 | 72.65 |
| 20.70 | 4.29 | 22.76 |
| 21.33 | 4.17 | 43.49 |
| 22.20 | 4.00 | 41.24 |
| 22.32 | 3.98 | 40.53 |
| 23.45 | 3.79 | 23.82 |
| 24.01 | 3.71 | 31.99 |
| 24.26 | 3.67 | 48.67 |
| 24.86 | 3.58 | 28.58 |
| 25.24 | 3.53 | 28.50 |
| 26.38 | 3.38 | 12.29 |
| 26.80 | 3.32 | 28.09 |
| 26.92 | 3.31 | 35.51 |
| 28.19 | 3.17 | 13.44 |
| 28.82 | 3.10 | 27.26 |
| 28.96 | 3.09 | 24.67 |
| 29.76 | 3.00 | 25.74 |
| 31.09 | 2.87 | 12.15 |
| 31.81 | 2.81 | 13.43 |
| 33.74 | 2.65 | 14.07 |
| 34.34 | 2.61 | 7.92 |
| 35.50 | 2.53 | 10.13 |
| 36.64 | 2.45 | 12.29 |
| 37.18 | 2.42 | 9.11 |
| 37.63 | 2.39 | 10.02 |
| 38.96 | 2.31 | 6.62 |

### Example 4

### Stability Assessment of Form B under Stress Conditions

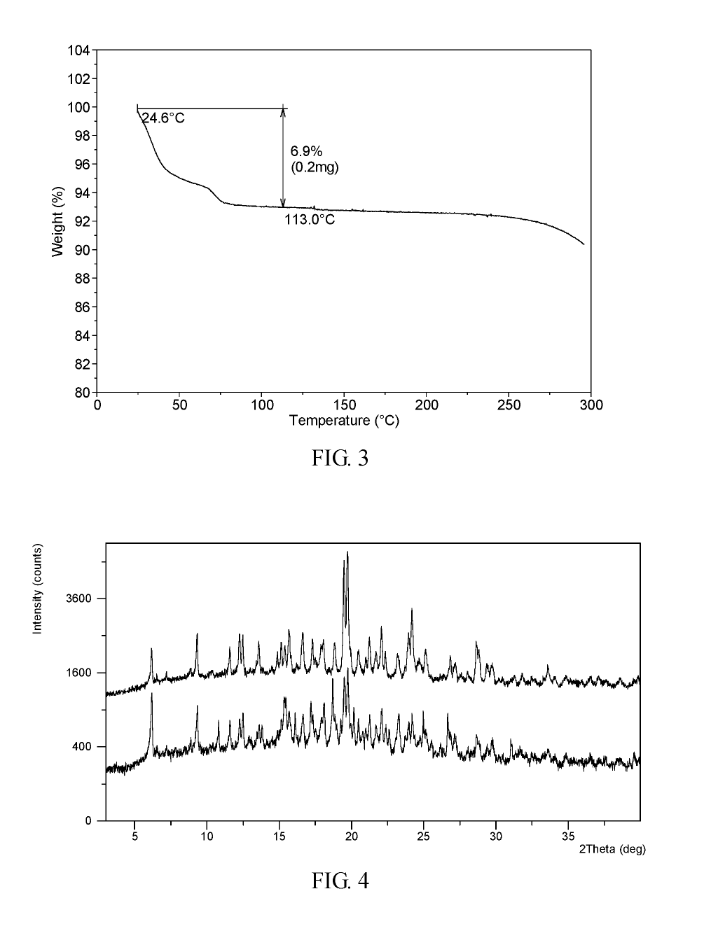

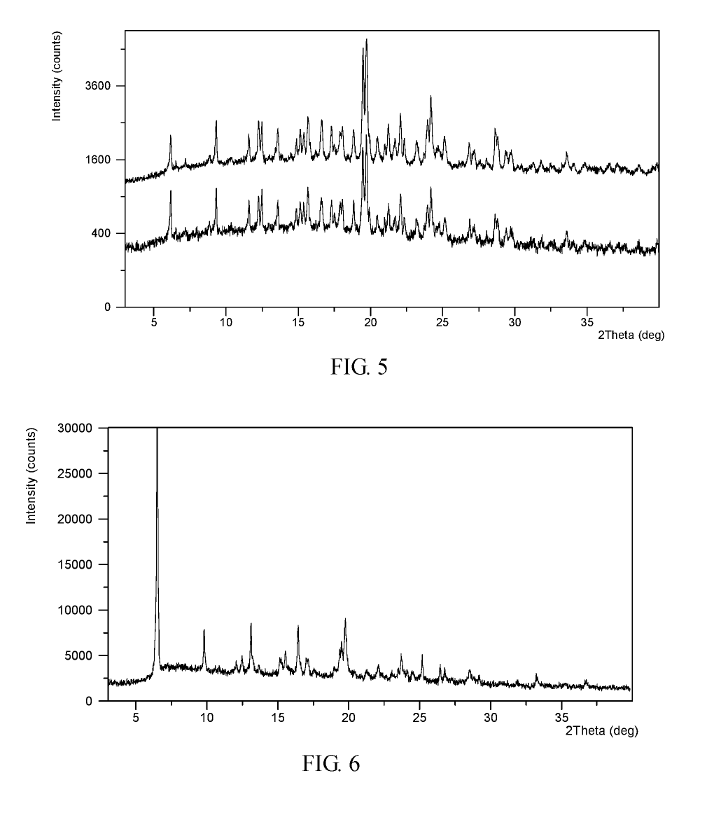

Two samples of canagliflozin Form B were stored under 25° C./60% RH and 40° C./75% RH, respectively, with dish open for 30 days. The solid samples were analyzed by XRPD. The XRPD patterns of the Form B sample before storage (top pattern) and of the Form B sample after stored under 25° C./60% RH for 30 days (bottom pattern) are displayed in FIG. 4; the XRPD diagrams of the Form B sample before storage (top pattern) and of the Form B sample after stored under 40° C./75% RH for 30 days (bottom pattern) are displayed in FIG. 5. The results of stability assessment tabulated in Table 4 suggest that Form B is stable under the stress conditions.

TABLE 4

| Initial Form | Conditions | Storage time | Final form |
|---|---|---|---|
| Form B | 25° C./60% RH | 30 days | Form B |
| (top pattern | | | (bottom pattern |
| in FIG. 4) | | | in FIG. 4) |
| Form B | 40° C./75% RH | 30 days | Form B |
| (top pattern | | | (bottom pattern |
| in FIG. 5) | | | in FIG. 5) |

### Example 5

### Kinetic Solubility Comparison Between Crystalline Form B of Canagliflozin and its Hemihydrate (WO 2008069327A1)

Kinetic solubility of canagliflozin in crystalline Form B and its hemihydrate form in fed state simulated intestinal fluid (FeSSIF) and fasted state simulated intestinal fluid (FaSSIF) were measured using the following procedures:

1. Weighed approximately 30 mg of compound into a tared 4 mL plastic vial and recorded the actual weight of the compound.

2. Added 3 mL of water or bio-relevant medium into each vial.

3. Covered the vials with caps and kept all the suspension samples stirring at RT using a rolling incubator with a speed of 25 r/min.

4. Sampled at 1 h, 4 h and 24 h respectively. About 0.6 mL aliquot of the suspension was transferred each time from solubility vial into a centrifuge filtration tube (pore size of 0.45 μm).

5. Centrifuged the filtration tubes in a speed of 8500 rpm for 3 minutes at RT, collected 0.2 mL supernatant for HPLC quantification determination and the rest of solution for pH measurement, and separated the solid for XRPD characterization.

The results displayed in Table 5 suggest that crystalline Form B of canagliflozin has higher solubility in comparison to it hemihydrate form as reported in WO2008069327A1.

TABLE 5

| | Time | FeSSIF | | FaSSIF | |
|---|---|---|---|---|---|
| | point | Form | WO2008069327A1 | Form | WO2008069327A1 |
| | (h) | B | hemihydrate | B | hemihydrate |
| Solu- | 1 | 4.72 | 3.34 | 0.50 | 0.36 |
| bility | 4 | 4.76 | 3.50 | 0.51 | 0.37 |
| at RT | 24 | 4.35 | 3.70 | 0.48 | 0.37 |
| (mg/ | | | | | |
| mL) | | | | | |

### Example 6

### Preparation of Crystalline Form C

In 5 mL of tetrahydrofuran/water (1:1, v/v) was dissolved 52.3 mg of canagliflozin, and the solution was filtered using a 0.45 μm Nylon filter. The filtrate was slowly evaporated to dryness, and the solid residue was dried in vacuum overnight. Form C was obtained after storing the solid under ambient conditions for 3 h. The XRPD pattern of the crystalline Form C produced in this Example is displayed in FIG. 6, the DSC thermogram is displayed in FIG. 7, and the TGA thermogram is displayed in FIG. 8. The XRPD data of the crystalline Form C are listed in Table 6.

TABLE 6

| 2 theta | d spacing | intensity % |
|---|---|---|
| 3.24 | 27.31 | 2.00 |
| 6.52 | 13.57 | 100.00 |
| 9.79 | 9.03 | 5.03 |
| 12.10 | 7.32 | 1.41 |
| 12.48 | 7.09 | 2.93 |
| 13.09 | 6.76 | 8.77 |
| 15.54 | 5.70 | 4.14 |
| 16.40 | 5.41 | 6.09 |
| 17.15 | 5.17 | 1.09 |
| 19.36 | 4.58 | 2.71 |
| 19.71 | 4.50 | 5.62 |
| 21.46 | 4.14 | 0.93 |
| 22.14 | 4.01 | 0.80 |
| 22.96 | 3.87 | 1.00 |
| 23.73 | 3.75 | 2.05 |
| 25.15 | 3.54 | 4.97 |
| 26.40 | 3.38 | 2.18 |
| 29.21 | 3.06 | 0.83 |
| 31.94 | 2.80 | 0.43 |
| 33.19 | 2.70 | 2.67 |
| 35.28 | 2.54 | 0.55 |
| 36.63 | 2.45 | 1.19 |

### Example 7

### Preparation of Crystalline Form C

In 40 mL mixture of tetrahydrofuran/water (1:3, v/v) was dissolved 508.1 mg of canagliflozin. The solution was heated to 60° C., and subsequently cooled to ambient temperature at 1° C./min while stirring. The solids were collected and stored under 22.5% RH for 1 week to obtain crystalline Form C. The XRPD data are displayed in Table 7.

TABLE 7

| 2 theta | d spacing | intensity % |
|---|---|---|
| 3.26 | 27.08 | 2.38 |
| 6.49 | 13.63 | 100.00 |
| 7.33 | 12.06 | 4.50 |
| 9.72 | 9.10 | 16.16 |
| 12.25 | 7.23 | 13.86 |
| 12.97 | 6.83 | 18.76 |
| 13.73 | 6.45 | 4.58 |
| 15.26 | 5.81 | 13.42 |
| 15.85 | 5.59 | 4.48 |
| 16.26 | 5.45 | 10.33 |
| 17.04 | 5.20 | 4.83 |
| 18.36 | 4.83 | 4.04 |
| 18.98 | 4.67 | 11.32 |
| 19.46 | 4.56 | 10.74 |
| 19.76 | 4.49 | 14.50 |
| 20.21 | 4.39 | 6.15 |
| 20.61 | 4.31 | 3.82 |
| 21.10 | 4.21 | 4.83 |
| 21.82 | 4.07 | 5.28 |
| 22.41 | 3.97 | 4.30 |
| 22.75 | 3.91 | 6.36 |
| 22.97 | 3.87 | 5.25 |
| 23.27 | 3.82 | 8.06 |
| 23.81 | 3.73 | 4.37 |
| 24.02 | 3.71 | 7.72 |
| 24.71 | 3.60 | 10.93 |
| 25.20 | 3.53 | 3.40 |
| 25.83 | 3.45 | 6.20 |
| 26.50 | 3.36 | 1.04 |
| 27.17 | 3.28 | 1.84 |
| 27.99 | 3.19 | 2.43 |
| 28.65 | 3.12 | 6.16 |
| 29.59 | 3.02 | 1.52 |
| 31.01 | 2.88 | 1.96 |
| 31.79 | 2.81 | 2.44 |
| 32.74 | 2.74 | 3.94 |
| 34.15 | 2.63 | 1.60 |
| 34.96 | 2.57 | 2.38 |
| 36.16 | 2.48 | 0.71 |
| 37.27 | 2.41 | 2.02 |

### Example 8

### Preparation of Crystalline Form D

Canagliflozin Form C (5 mg) was heated to 80° C. under nitrogen protection to obtain crystalline Form D. The XRPD pattern of Form D produced in this Example is dispalyed in FIG. 9. The XRPD data are listed in Table 8.

TABLE 8

| 2 theta | d spacing | intensity % |
|---|---|---|
| 3.37 | 26.20 | 0.96 |
| 6.11 | 14.46 | 2.16 |
| 6.79 | 13.02 | 100.00 |
| 10.20 | 8.67 | 5.26 |
| 11.30 | 7.83 | 2.73 |
| 11.52 | 7.68 | 2.73 |
| 12.49 | 7.09 | 4.66 |
| 13.65 | 6.49 | 27.75 |
| 14.42 | 6.14 | 4.22 |
| 15.59 | 5.68 | 6.15 |
| 16.06 | 5.52 | 6.40 |
| 16.50 | 5.37 | 8.94 |
| 17.09 | 5.19 | 14.67 |
| 17.54 | 5.06 | 6.55 |
| 18.56 | 4.78 | 7.65 |
| 19.18 | 4.63 | 12.18 |
| 19.58 | 4.53 | 16.29 |
| 19.76 | 4.49 | 12.93 |
| 20.55 | 4.32 | 16.04 |
| 21.13 | 4.20 | 6.00 |
| 22.07 | 4.03 | 6.02 |
| 22.95 | 3.88 | 9.49 |
| 23.20 | 3.83 | 6.39 |
| 23.59 | 3.77 | 4.54 |
| 24.42 | 3.64 | 7.23 |
| 24.84 | 3.58 | 2.65 |
| 27.87 | 3.20 | 2.31 |
| 28.28 | 3.16 | 1.52 |
| 31.07 | 2.88 | 1.46 |
| 32.14 | 2.79 | 0.63 |
| 33.30 | 2.69 | 0.81 |

### Example 9

### Comparison of Kinetic Solubility Between Crystalline Form C and Hemihydrate Form (WO2008069327A1) of Canagliflozin

Kinetic solubility of canagliflozin Form C and hemihydrate in fed state simulated intestinal fluid (FeSSIF) and fasted state simulated intestinal fluid (FaSSIF) were measured using the following procedures:

1. Weighed approximately 30 mg of compound into a tared 4 mL plastic vial and recorded the actual weight of the compound.

2. Added 3 mL of water or bio-relevant medium into each vial.

3. Covered the vials with caps and kept all the suspension samples stirring at RT using a rolling incubator with a speed of 25 r/min.

4. Sampled at 1 h, 4 h and 24 h respectively. About 0.6 mL aliquot of the suspension was transferred each time from solubility vial into a centrifuge filtration tube (pore size of 0.45 μm).

5. Centrifuged filtration tubes in a speed of 8500 rpm for 3 minutes at RT, collected 0.2 mL supernatant for HPLC quantification determination and the rest of solution for pH measurement, and separated the solid for XRPD characterization.

The results displayed in Table 9 suggest that crystalline Form C has higher solubility than the hemihydrate form (WO2008069327A1).

TABLE 9

| | Time | FeSSIF | | FaSSIF | |
|---|---|---|---|---|---|
| | point | Form | WO2008069327A1 | Form | WO2008069327A1 |
| | (h) | C | hemihydrate | C | hemihydrate |
| Solu- | 1 | 4.60 | 3.34 | 0.41 | 0.36 |
| bility | 4 | 4.70 | 3.50 | 0.44 | 0.37 |
| (mg/ | 24 | 4.50 | 3.70 | 0.42 | 0.37 |
| mL) | | | | | |

The foregoing examples and description of the preferred embodiments should be taken as illustrating, rather than as limiting, the present invention as defined by the claims. As will be readily appreciated by a person skilled in the art, numerous variations and combinations of the features set forth above can be utilized without departing from the present invention as set forth in the claims. All such variations are intended to be included within the scope of the following claims. All references cited herein are incorporated by reference in their entireties.

What is claimed is:

1. A crystalline form of canagliflozin or hydrate thereof, designated as Form B, having a powder X-ray diffraction pattern comprising the following 2θ values measured using Cu Kα radiation: 6.3°±0.2°, 9.4°±0.2°, and 12.6°±0.2°.

2. The crystalline Form B of claim 1, wherein the X-ray diffraction pattern further comprises the following 2θ values measured using Cu Kα radiation: 11.7°±0.2°, 16.9°±0.2°, and 19.9°±0.2°.

3. The crystalline Form B of claim 1 or 2, wherein the X-ray diffraction pattern further comprises the following 2θ values measured using Cu Kα radiation: 18.2°±0.2°, 22.3°±0.2°, 24.4°±0.2°, and 28.9°±0.2°.

4. The crystalline Form B of claim 1, having an X-ray diffraction pattern substantially as depicted in FIG. 1.

5. The crystalline Form B of claim 1, having a differential scanning calorimetric thermogram substantially as depicted in FIG. 2.

6. A process for the preparation of crystalline Form B of canagliflozin, comprising: dissolving canagliflozin in a solvent system comprising water and an organic solvent; and evaporating the solvents in a controlled manner to precipitate canagliflozin as a crystalline solid.

7. The process of claim 6, wherein the organic solvent is selected from the group consisting of methanol, ethanol, 2-propanol, acetonitrile, acetone, and tetrahydrofuran.

8. The process of claim 6, wherein said organic solvent is ethanol or tetrahydrofuran.

9. The process of any one of claims 6 to 8, wherein the volume ratio of water to said organic solvent is from 1:10 to 10:1.

10. The process of any one of claims 6 to 8, wherein said volume ratio of water to organic solvent is 1:1.

11. A crystalline form of canagliflozin or hydrate thereof, designated as Form C, having a powder X-ray diffraction pattern comprising the following 2θ values measured using Cu Kα radiation: 6.5°±0.2°, 9.8°±0.2°, and 16.4°±0.2°.

12. The crystalline Form C of claim 11, wherein the X-ray diffraction pattern further comprises the following 2θ values measured using Cu Kα radiation: 13.1°±0.2°, 19.8°±0.2°, and 23.7°±0.2°.

13. The crystalline Form C of claim 11 or 12, wherein the X-ray diffraction pattern further comprises the following 2θ values measured using Cu Kα radiation: 17.1°±0.2°, 19.5°±0.2 °, 25.2°±0.2°, and 26.5°±0.2°.

14. The crystalline Form C of claim 11, having an X-ray diffraction pattern substantially as depicted in FIG. 6.

15. The crystalline form C of claim 11, having a differential scanning calorimetry thermogram substantially as depicted in FIG. 7.

16. The crystalline Form C of claim 11, having a thermal gravimetric analysis thermogram substantially as depicted in FIG. 8.

17. A process for preparation of Form C, comprising: dissolving canagliflozin in a mixed solvent system comprising water and tetrahydrofuran; evaporating substantially all the solvents to dryness to form a solid; and sweeping the solid using air or N₂ to form said crystalline Form C.

18. A crystalline form of canagliflozin or hydrate thereof, designated as Form D, having a powder X-ray diffraction pattern comprising the following 2θ values measured using Cu Kα radiation: 6.8°±0.2°, 13.6°±0.2°, and 20.5°±0.2°.

19. The crystalline Form D of claim 18, wherein the X-ray diffraction pattern further comprises the following 2θ values measured using Cu Kα radiation: 17.1°±0.2°, 19.2°±0.2°, and 22.9°±0.2°.

20. The crystalline Form D of claim 18 or 19, wherein the X-ray diffraction pattern further comprises the following 2θ values measured using Cu Kα radiation: 10.2°±0.2°, 16.5°±0.2°, 18.5°±0.2°, and 24.4°±0.2°.

21. The crystalline Form D of claim 18, having an X-ray diffraction pattern substantially as depicted in FIG. 9.

22. A process for the preparation of the Form D of any one of claims 18 to 21, comprising heating a crystalline Form C of canagliflozin to 50° C-90° C.

23. A pharmaceutical composition comprising one or more of crystalline Forms B, C, and D of canagliflozin or a hydrate thereof, and a pharmaceutically acceptable carrier, wherein said crystalline Form B has a powder X-ray diffraction pattern comprising the following 2θ values measured using Cu Kα radiation: 6.3°±0.2°, 9.4°±0.2°, and 12.6°±0.2°; said crystalline Form C has an powder X-ray diffraction pattern comprising the following 2θ values measured using Cu Kα radiation: 6.5°±0.2°, 9.8°±0.2°, and 16.4°±0.2°; and said crystalline Form D has a powder X-ray diffraction pattern comprising the following 2θ values measured using Cu Kα radiation: 6.8°±0.2°, 13.6°±0.2°, and 20.5°±0.2°.

24. The pharmaceutical composition of claim 23, wherein the X-ray diffraction pattern of said crystalline Form B further comprises the following 2θ values measured using Cu Kα radiation: 11.7°±0.2°, 16.9°±0.2°, and 19.9°±0.2°.

25. The pharmaceutical composition of claim 23 or 24, wherein the X-ray diffraction pattern of said crystalline Form B further comprises the following 2θ values measured using Cu Kα radiation: 18.2°±0.2°, 22.3°±0.2°, 24.4°±0.2°, and 28.9°±0.2°.

26. The pharmaceutical composition of claim 23 or 24, wherein said crystalline Form B has a melting point around 92.5° C. as measured by DSC.

27. The pharmaceutical composition of claim 23, wherein the X-ray diffraction pattern of said crystalline Form B has an X-ray diffraction pattern substantially as depicted in FIG. 1.

28. The pharmaceutical composition of claim 23, wherein the X-ray diffraction pattern of said crystalline Form C further comprises the following 2θ values measured using Cu Kα radiation: 13.1°±0.2°, 19.8°±0.2°, and 23.7°±0.2°.

29. The pharmaceutical composition of claim 23 or 28, wherein the X-ray diffraction pattern of said Form C further comprises the following 2θ values measured using Cu Kα radiation: 17.1°±0.2°, 19.5°±0.2°, 25.2°±0.2°, and 26.5°±0.2°.

30. The pharmaceutical composition of claim 23, wherein the X-ray diffraction pattern of said Form C has an X-ray diffraction pattern substantially as depicted in FIG. 6.

31. The pharmaceutical composition of claim 23, wherein the X-ray diffraction pattern of said Form C has a differential scanning calorimetry thermogram substantially as depicted in FIG. 7.

32. The pharmaceutical composition of claim 23, wherein the X-ray diffraction pattern of said Form C has a thermal gravimetric analysis thermogram substantially as depicted in FIG. 8.

33. The pharmaceutical composition of claim 23, wherein the X-ray diffraction pattern of said crystalline Form D further comprises the following 2θ values measured using Cu Kα radiation: 17.1°±0.2°, 19.2°±0.2°, and 22.9°±0.2°.

34. The pharmaceutical composition of claim 23 or 33, wherein the X-ray diffraction pattern of said crystalline Form D further comprises the following 2θ values measured using Cu Kα radiation: 10.2°±0.2°, 16.5°±0.2°, 18.5°±0.2°, and 24.4°±0.2°.

35. The pharmaceutical composition of claim 23, wherein said crystalline Form D has an X-ray diffraction pattern substantially as depicted in FIG. 9.

36. A method of treating or delaying the progression or onset of a disease or disorder in connection with activity of a sodium-glucose transport (SGLT) protein, comprising administering to a subject in need thereof a pharmaceutical composition comprising any one of crystalline Forms B, C, and D of canagliflozin or hydrate or combination thereof.

37. The method of claim 36, wherein said disease or disorder is selected from the group consisting of diabetes mellitus, diabetic retinopathy, diabetic neuropathy, diabetic nephropathy, delayed wound healing, insulin resistance, hyperglycemia, hyperinsulinemia, elevated blood levels of fatty acids, elevated blood levels of glycerol, hyperlipidemia, obesity, hypertriglyceridemia, Syndrome X, diabetic complications, atherosclerosis, and hypertension.

38. Use of crystalline Form B, C, or D of canagliflozin, or a combination thereof, in the manufacture of a medicament for treating or delaying the progression or onset of a disease or disorder in connection with activity of a sodium-glucose transport (SGLT) protein.

39. The use of claim 38, wherein said disease or disorder is selected from the group consisting of diabetes mellitus, diabetic retinopathy, diabetic neuropathy, diabetic nephropathy, delayed wound healing, insulin resistance, hyperglycemia, hyperinsulinemia, elevated blood levels of fatty acids, elevated blood levels of glycerol, hyperlipidemia, obesity, hypertriglyceridemia, Syndrome X, diabetic complications, atherosclerosis, and hypertension.
